(19) United States
(12) Patent Application Publication
(10) Pub. No.: US 2006/0134679 A1
(43) Pub. Date: Jun. 22, 2006

(54) Methods and compositions for acquiring information from unstretched polymer conformations

(75) Inventor: Jonathan W. Larson, New Ipswich, NH (US)

Correspondence Address: WOLF GREENFIELD & SACKS, PC;FEDERAL RESERVE PLAZA, 600 ATLANTIC AVENUE, BOSTON, MA, 02210-2206, US

(73) Assignee: U.S. Genomics, Inc., Woburn, MA (US)

(21) Appl. No.: 11/311,696

(22) Filed: Dec. 19, 2005

Related U.S. Application Data
(60) Provisional application No. 60/636,940, filed on Dec. 17, 2004.

(51) Int. Cl. C12Q 1/68

(52) U.S. Cl. 435/6

(57) ABSTRACT

The invention relates to the methods for analyzing polymers that are in unstretched conformations. In particular, the polymers such as nucleic acids may be present in hairpin conformations.

### RELATED APPLICATIONS

This application claims priority to U.S. provisional application having Ser. No. 60/636,940, entitled “METHODS AND COMPOSITIONS FOR ACQUIRING INFORMATION FROM UNSTRETCHED POLYMER CONFORMATIONS”, filed on Dec. 17, 2004, the entire contents of which are incorporated by reference herein.

### FIELD OF THE INVENTION

The invention relates broadly to the field of polymer analysis, such as nucleic acid analysis.

### BACKGROUND OF THE INVENTION

Polymers naturally tend toward an unstretched conformation. However, polymer analysis preferably uses polymers in a linearized and stretched conformation. In some prior art methods, it is practically essential that polymers be stretched before and/or during analysis in order to obtain meaningful linear or contiguous sequence information. Information generated from unstretched polymers is usually considered unusable and thus is discarded because the relative position and order of bound probes cannot be discerned. Since every polymer is equally likely to adopt an unstretched conformation, some fraction of polymers goes unanalyzed. If the polymers being analyzed are rare, such as for example in a forensic analysis or in mRNA transcript analysis, larger starting polymer population sizes are required or alternatively important information is not attained.

Accordingly, there exists a need for methods that are able to measure and usefully discern sequence information from unstretched polymers, such as nucleic acids.

### SUMMARY OF THE INVENTION

The invention relates in part to the observation that even under conditions designed for optimally stretching polymers, such as microfluidic applications, a significant proportion of polymers continue to exist quite stably in unstretched forms. The predominant unstretched form observed in certain microfluidic methods has been a hairpin conformation, and even more predominantly a single hairpin conformation (i.e., a single 180° bend). It has not been heretofore appreciated that sequence (e.g., identity) information can be obtained from such unstretched polymers. The invention is based in part on the recognition that information from unstretched polymers, and particularly single hairpin polymers, is usable. The invention therefore provides methods for attaining such information from polymers in unstretched conformations.

Thus, in one aspect, the invention provides a method for analyzing a polymer in an unstretched conformation comprising identifying a single hairpin polymer in a polymer population based on a measured length that is shorter than a contour length, and comparing a probe binding profile of the single hairpin polymer to a hairpin dataset, wherein the polymer is labeled with a backbone label.

In one embodiment, the measured length is determined based on transit time of the polymer between two positions wherein the polymer is traveling at a known velocity. The two positions may be two interrogation zones or two detection zones. In another embodiment, the contour length is determined by measuring total backbone label signal intensity for the polymer. In another embodiment, the total backbone label signal intensity is total integrated signal intensity.

In another aspect, the invention provides a method for analyzing a polymer in an unstretched conformation comprising determining total integrated backbone label signal intensity of a polymer as an indicator of contour length wherein the polymer is labeled with a backbone label, determining measured length based on transit time of the polymer between two positions wherein the polymer is traveling at a known velocity, comparing contour length and measured length wherein a measured length that is shorter than a contour length is indicative of an unstretched polymer, determining a probe binding profile of the unstretched polymer, and processing the probe binding profile. The two positions may be two interrogation zones or two detection zones.

In one embodiment, processing the probe binding profile comprises derivation of de novo sequence information from the profile. In another embodiment, processing the probe binding profile comprises comparing the probe binding profile to a hairpin dataset.

These aspects share various embodiments and these are recited below.

In one embodiment, the hairpin dataset contains forward and reverse orientation probe binding profiles for a hairpin polymer. In another embodiment, the method further comprises re-orienting probe binding profiles according to leading edge or trailing edge high signal intensity backbone regions.

In one embodiment, the polymer is a nucleic acid such as but not limited to a DNA or RNA.

In one embodiment, the probe binding profile (or pattern) is a sequence specific probe binding profile. In one embodiment, sequence specific probes consisting of the same sequence are labeled identically. In another embodiment, sequence specific probes consisting of a different sequence are labeled differentially. In another embodiment, at least one of the sequence specific probes is unique to an organism. In yet another embodiment, the sequence specific probes are chosen to yield a unique probe binding profile indicative of an organism.

In one embodiment, the hairpin dataset comprises signals from hairpin configurations of pathogen derived polymers. In another embodiment, the hairpin dataset comprises signals from hairpin configurations of polymers from a single pathogen. In a related embodiment, the pathogen derived polymers are derived from biohazardous pathogens. In another embodiment, the hairpin dataset comprises signals from hairpin configurations of human derived polymers. In a related embodiment, the human derived polymers are genomic DNA.

These and other embodiments of the invention will be described in greater detail herein. Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE DRAWINGS

The accompanying drawings are not intended to be drawn to scale. For purposes of clarity, not every component may be labeled in every drawing.

FIG. 1 is a schematic illustrating potential, and in some instances predominant, polymer conformations following sudden elongation flow. Illustrated are a stretched, linear conformation and an unstretched, single hairpin conformation. If such polymers are labeled with a backbone label, the polymer in the stretched conformation will emit signal from such label essentially equally along its length. The polymer in the hairpin conformation will have a low signal and a high signal region corresponding to its stretched and hairpinned regions.

FIG. 2 is a schematic illustrating the positioning of three probes on a polymer as a function of the conformation of the polymer. In the stretched, linear polymer, the three probes are spatially separated and would be detected by a detector as three separate signals or events each indicative of a probe bound to the polymer at that particular location. In the unstretched, single hairpin polymer, the relative positioning of the probes is different. Specifically in the example shown, two probes are directly overlayed. As discussed for FIG. 1, the hairpinned polymer can be distinguished from the linear stretched polymer based on length (using signal intensities). The probes may each be unique for the polymer of interest or their combined binding (and optionally resultant pattern) may be unique for the polymer. The use of such probes can provide identity and/or sequence information of the polymer.

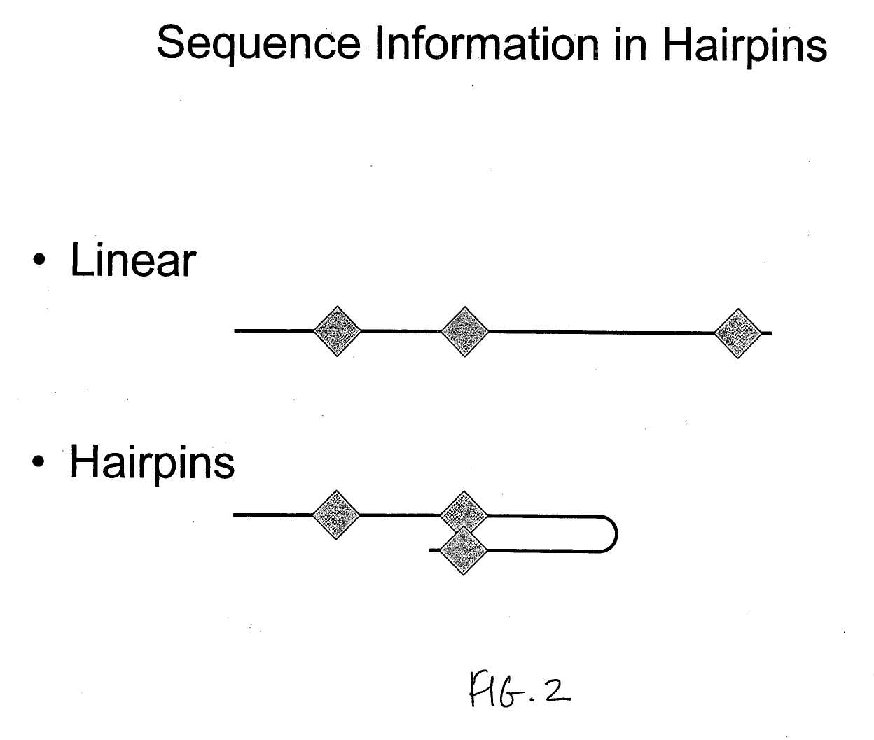

FIG. 3 is a schematic illustrating certain possible conformations of a single hairpin polymer. The top two arrangements illustrate that the identical single hairpin polymer may enter a detection zone in one of two orientations, and thus may be read as two different polymers. The invention provides a mechanism for identifying such conformations as simply reverse orientations of a single polymer using backbone intensity levels to distinguish between stretched and hairpinned regions. The bottom examples illustrate the conformations possible if the hairpin forms at a different location along the polymer length, with all polymers oriented with the hairpin in the leading edge. Although the polymer in both examples is the same, the readouts will be different. Using prior art methods, one might consider that these polymers are different from each other due to altered probe binding profile (or pattern). The invention contemplates a method for determining whether two or more polymers with different probe binding profiles are actually the same polymer. The profile may be compared with a known fingerprint (or a profile of a known polymer). Thus, one particularly useful application of the methods provided herein is the ability to identify the presence of a polymer in a sample such as for example in a biohazard agent detection application.

It is to be understood that the drawings are not required for enablement of the invention.

### DETAILED DESCRIPTION OF THE INVENTION

The invention therefore recognizes and provides means for analyzing as many polymers within a population independent of their conformation. To this end, the invention makes polymer analysis more efficient since data can be obtained from a greater proportion of polymers.

The invention is based partly on the recognition that systems which attempt to linearize and stretch polymers do so at less than perfect efficiency under certain conditions. Some fraction of polymers may still exist in an unstretched conformation even under conditions designed to stretch polymers maximally. The most predominant unstretched polymer conformation is that of a hairpin, and even more predominant a single hairpin. An unstretched conformation makes polymer analysis difficult if not impossible using prior art methods.

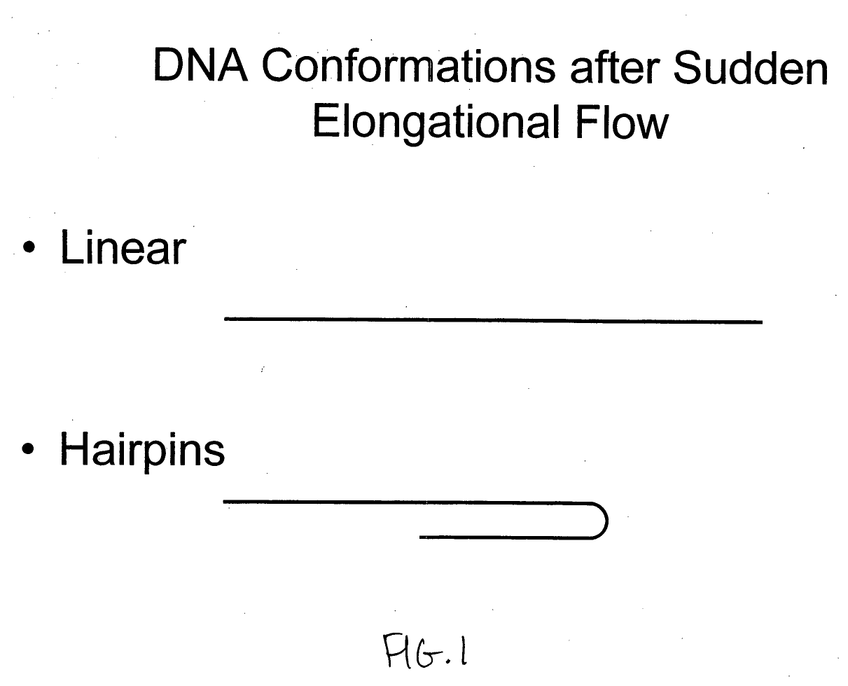

The invention however provides methods for analyzing unstretched polymers and preferably single hairpin polymers, thereby overcoming this prior art limitation. A stretched polymer is shown in FIG. 1, top panel. An unstretched polymer is a linear polymer in any other conformation. Similarly, an unstretched conformation is any conformation other than a stretched conformation as shown in FIG. 1. It may include the conformation of compacted polymers, bent polymers, hairpin polymers, and the like. It may also encompass polymer conformations having bends of less than or more than 180°. Preferably, the unstretched polymer is a hairpin polymer and more preferably it is a single hairpin polymer.

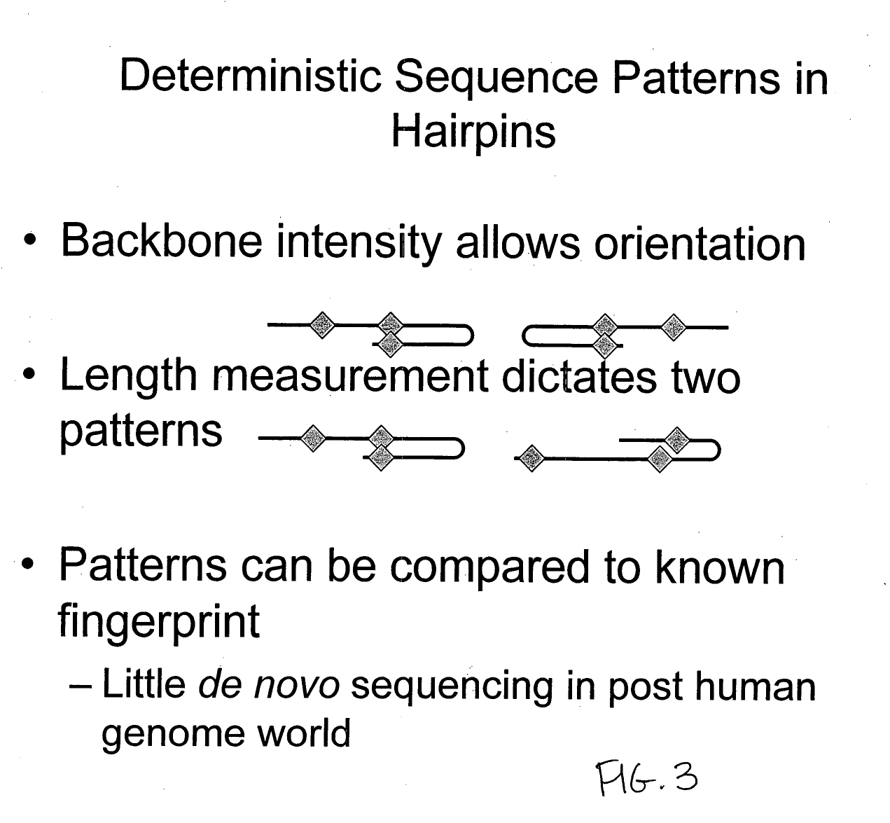

A single hairpin conformation is illustrated in the Figures. A hairpin polymer is a linear polymer that has at least one 180° bend in it. A single hairpin polymer is a linear polymer that has only one 180° bend. The bend can occur anywhere along the length of the polymer, although the stability of hairpin structures will vary depending on the location of the bend. For example, bends closer to the end of the polymer will generally be less stable and thus will tend to become stretched. Stability of a hairpin structure will also depend on the orientation of the hairpin relative to the flow in which the polymer is situated. Thus, hairpins at the leading edge of a polymer tend to be more stable than hairpins at the trailing edge of a polymer. These different structures are shown in FIG. 3, top panel. Due to flow resistance, there is a greater probability that the trailing edge hairpin will stretch out than will the leading edge hairpin. Thus, although any hairpin orientation and conformation is possible, some are more stable under the particular flow conditions and thus will predominate.

One aspect of the invention relates to the identification of unstretched polymers from a population of polymers. Stretched and unstretched polymers can be distinguished from each other in a number of ways, depending on the polymer and the labeling strategy.

One such way is to determine and compare the contour (or true) length and measured (or observed) length of the polymer, since a difference between these will indicate a potential unstretched conformation. The contour length represents the length of the polymer when it is in a stretched conformation. The contour length can be measured in a number of ways. In one particularly important embodiment, the contour length is measured by determining the total intensity associated with a polymer that is either intrinsically or extrinsically labeled with a detectable and preferably ubiquitous label, such as but not limited to a backbone label or stain. Preferably the label is chosen so that it provides substantially uniform labeling (and thus signal) along the length of the polymer. Accordingly, a relatively sequence non-specific backbone label or stain is suitable for this purpose. Alternatively, one or more sequence specific probes can also be used provided their complementary sequences are relatively ubiquitous and common so that the probe(s) binds almost contiguously along the length of the polymer (e.g., a two-mer or three-mer probe set encompassing every nucleotide pair or triplet combination, respectively). Due to the cost of synthesizing probes, it may some times be preferably to use a backbone label instead.

The polymer intensity for at least this embodiment of the invention refers to the total signal intensity deriving from the ubiquitous label (such as the backbone label). Although the polymer may also be bound to probes, it is expected that the signal from such probes will be distinguishable from the ubiquitous label. The total polymer intensity generally refers to the total integrated signal intensity (i.e., the area under the curve on a intensity versus time or distance histogram). This measurement is indicative of contour length (or true length) since it reflects the total amount of ubiquitous label on the polymer, a parameter that is dependent on true length and not polymer conformation. Thus, for example, a polymer of 1000 nucleotides in length is expected to have twice as much signal intensity associated with it than a polymer of 500 nucleotides in length.

The measured length is the length of the polymer as determined using a classical time, distance and velocity calculation. The method generally entails determining the time the polymer takes to travel between two positions such as two interrogation zones or two detection zones. An interrogation zone is the position at which the polymer (and/or any labels bound thereto) is interrogated. In some embodiments, this is equivalent to excitation with a laser, and thus the zone may be the location of polymer contact with an excitation laser. A detection zone is the position at which signals being emitted from the polymer (and/or labels bound thereto) are captured and forwarded to a detector (such as a photomultiplier tube, for example). The time it takes the polymer to travel between the two positions is referred to herein as the transit time. If the polymer is traveling at a known velocity, e.g., based on the flow dynamics of the system, then its length can be determined. As an example, a polymer labeled along its length (with for example a ubiquitous label) will pass a first detector at time zero and register a signal at that detector due to the presence of label at the front end of the polymer. The polymer will continue to move past the detector in a relatively linear manner until the last detectable label at the back end of the polymer is detected by the same detector. The time between the detection of the front end signal and the back end signal will be the transit time, in this embodiment. It should be understood that this distance information provided by this analysis will reflect the conformation of the polymer. For example, assume two polymers of the same contour (or true) length are being analyzed, the first of which is in a stretched conformation and the second of which is in a hairpin conformation. The first one will register a longer period of time between the time the front end signal is detected and the time the back end signal is detected. The second one will read out a shorter period of time between the time the front end signal is detected and the back end signal is detected because it is effectively shortened by the length of the hairpin. Thus, in a method capable of determining a contour length and a measured length for each polymer, a difference between these two parameters (i.e., a measured length that is shorter than the contour length) is indicative of a hairpin conformation.

It is also possible to determine the average intensity signal for a polymer. The average intensity signal for a polymer is the average amount of intensity per binning event (e.g., per timed window for data capture). A stretched polymer will register the same value for this parameter regardless of its length provided that every polymer is relatively uniformly labeled. An unstretched polymer however will register a higher value for this parameter for two reasons. First, binning events that capture signal from the hairpin will have higher intensities due to up to double the number of signals in this region of the polymer. Second, the number of binning events needed to capture the entirety of the polymer is lower since the measured (or observed) length of the polymer is shorter than the contour (or true) length of the polymer. If one plots out averaged intensity (i.e., intensity per binning event) versus total integrated intensity (or contour or true length), then one finds that the polymer data for a population of polymers exists in a hyperbola function. A large subset of polymers reside at a position associated with a stretched conformation. Polymers residing at positions of greater averaged intensity and shorter length are indicative of unstretched conformations, including hairpin polymers. One of ordinary skill in the art will be able to manipulate these various parameters based on the teachings provided herein in order to distinguish a stretched from an unstretched polymer. Thus, the invention envisions and embraces various combinations and/or permutations of these parameters for this purpose.

Once a polymer is identified as being in an unstretched conformation, it can then be more closely analyzed. Processing the probe binding profile as used herein refers to analyzing and/or comparing (including contrasting) the probe binding profile of the “test” polymer with one or more “control” hairpin datasets. The control hairpin datasets may be previously established datasets for known polymers, and can be used to determine the identity of the test polymer. The probe binding profile may be represented by the backbone label versus length (or time) histogram, and this may be retrieved and further analyzed or processed. As another example, the probe signal versus length (or time) histogram optionally or alternatively can be retrieved and further analyzed or processed. Thus, the polymer has been exposed to (or contacted with) a probe that binds to the polymer preferably in a sequence or structure specific manner (unlike the backbone label previously discussed). Binding of one or more such probes and the relative position of such bound probes on a polymer can be used to determine the nature and source of the polymer, among other things. Thus, the invention contemplates contacting a polymer or polymer population with one or more sequence specific probes under conditions that allow the probes to bind in a sequence or structure specific manner. The sequence specific probes are preferably designed and/or selected to provide nature, identity and/or source information regarding the polymer. For example, the probes may be chosen such that one, some or all are unique to a particular genome (such as for example the genome of a pathogen such as a biohazardous pathogen). This is particularly useful if one of the goals of the ultimate analysis is to determine if a particular organism such as a pathogen is present in a sample being tested. The probes may also be selected to distinguish between genomes of different subjects of a given species, such as may be useful in for example a forensic DNA analysis. If one is interested in knowing if a sample contains DNA of a given individual (for example, someone already in a database, whether his or her identity is known), then one can devise one or more probes that will uniquely identify (e.g., by pattern of labeling) the genetic material of that individual and then use those probes on the sample to determine if it contains such genetic material. In yet another example, probes may be selected as uniquely identifying a genetic mutation such as a deletion, substitution, addition, etc. that is associated with a particular condition or disease state. An example of such a dataset is shown in FIG. 3 which demonstrates at least three different conformations that can be assumed by a given single hairpin polymer, albeit with two differentially placed hairpin structures. The dataset may contain in some embodiments forward (leading edge) and reverse (trailing edge) orientations for each hairpin polymer as well as various hairpin structures differing according to the location of the hairpin (see FIG. 3). The dataset may be manipulated in order to orient all polymer profiles in one direction. Whether the hairpin is present in the leading or the trailing edge can be determined by the region of high signal intensity backbone regions since hairpin regions will emit higher signal intensity than will stretched regions.

The pattern of probes bound to the polymer is referred to as the probe binding pattern or profile and it can be compared to datasets of probe binding patterns or profiles. The datasets can be simulations of every hairpin conformation a given polymer may assume (including those thought to be less stable), or they may comprise solely a subset of such conformations. The dataset preferably represents the probe binding pattern expected for each hairpin conformation. Thus, by comparing the experimental probe binding patterns with the simulated dataset, it will be possible to determine if the experimental probe binding pattern has a match in the dataset. This in turn will reveal information relating to nature, identity and/or source of the polymer, among other things.

The invention further contemplates a screening process that may be incorporated at any point in the analysis. For example, if the analysis involves identifying a polymer from a particular pathogen based on binding of three distinct sequence specific probes, then the method may incorporate a step in which polymers that are not labeled with at least those probes are removed and not subject to further analysis. In this embodiment, it is preferable to label each of the probes uniquely so that the presence of each is indicated by a separate readout. Signals from such uniquely labeled polymers may then be collected and analyzed separately form other polymer signals.

It is to be understood that the method contemplates analysis of one or more specific polymers from particular organism in order to conclude that a positive event has occurred. For example, the method may rely on the detection of two particular polymer regions within the genome of a pathogen in order to conclude that the pathogen is present in a sample. Given the downstream consequences of making such a determination, it is expected that one practicing the invention for this purpose will avoid as much as possible false positives by detecting as many polymers as is necessary.

Polymer analysis generally refers to obtaining sequence and/or structural information from a polymer. This information can be obtained at various levels of resolution. Polymer analysis embraces de novo compilation of sequence information based on, for example, the presence and relative location of sequence specific probes on the polymer. It also embraces comparison of a probe binding profile to a standard, such as a previously established map. Thus, for example, a probe binding profile for a nucleic acid can be compared to a previously established genetic map for, for example, an organism in order to determine the identity and/or origin of the nucleic acid. This latter scenario illustrates how nucleic acids can be used to detect organisms such as for example biohazardous agents. This latter approach also finds application in haplotyping and genotyping scenarios. Comparison based analyses may be performed with unstretched nucleic acids potentially just as well as with stretched nucleic acids.

Thus, the invention contemplates in one embodiment polymer analysis via comparison with previously established datasets such as those discussed above, genome maps, and the like. These may involve the human genome (or fragments thereof) or other genomes (or fragments thereof), such as but not limited microbial genomes. Such genetic maps can be accessed at for example the NCBI website. Genetic maps can also be constructed by one practicing the invention prior to the analysis described herein. For example, one may establish a map with a sufficient number of unique probes (or unique probe binding profile or pattern) to be used as a standard against which test samples and test (or target) polymers may be compared. As will be understood, a greater level of detail in a binding pattern or profile will be more likely to discriminate between genetic maps.

Biohazardous agents of particular interest include but are not limited to CDC Category A, B and C agents. CDC Category A agents include Bacillus anthracis (otherwise known as anthrax), Clostridium botulinum (causative agent for botulism), Yersinia pestis (causative agent for the plague), variola major (causative agent for small pox), Francisella tularensis (causative agent for tularemia), and viral hemorrhagic fever causing agents such as filoviruses Ebola and Marburg and arenaviruses such as Lassa, Machupo and Junin.

CDC Category B agents include Brucellosis (Brucella species), Clostridium perfringens, food safety threats such as Salmonella species, E. coli and Shigella, Glanders (Burkholderia mallei), Melioidosis (Burkholderia pseudomallei), Psittacosis (Chlamydia psittaci), Q fever (Coxiella burnetii), Staphylococcal enterotoxin B, Typhus fever (Rickettsia prowazekii), viral encephalitis (alphaviruses, e.g., Venezuelan equine encephalitis, eastern equine encephalitis, western equine encephalitis), and water safety threats such as e.g., Vibrio cholerae and Cryptosporidium parvum.

CDC Category C agents include emerging infectious agents such as Nipah virus and hantavirus.

Other pathogens that can be detected using the methods of the invention include Gonorrhea, H. pylori, Staphylococcus spp., Streptococcus spp. such as Streptococcus pneumoniae, Syphilis; viruses such as SARS virus, Hepatitis virus, Herpes virus, HIV virus, West Nile virus, Influenza virus, poliovirus, rhinovirus; parasites such as Giardia, and Plasmodium malariae (malaria); and mycobacteria such as M. tuberculosis.

A “polymer” as used herein is a compound having a linear (i.e., contiguous) backbone of individual units which are linked together by linkages. The term “backbone” is given its usual meaning in the field of polymer chemistry. The polymers may be homogeneous or heterogeneous in backbone composition. The polymers may be, for example, nucleic acids, polypeptides, polysaccharides or carbohydrates. A polypeptide as used herein is a biopolymer comprised of linked amino acids. In the most preferred embodiments, the polymer is a nucleic acid.

As used herein with respect to linked units of a polymer, “linked” or “linkage” means two entities bound to one another by any physicochemical means. Any linkage known to those of ordinary skill in the art, covalent or non-covalent, is embraced. Natural linkages, which are those ordinarily found in nature connecting the individual units of a particular polymer, are most common. Natural linkages include, for instance, amide, ester and thioester linkages. The individual units of a polymer analyzed by the methods of the invention may be linked, however, by synthetic or modified linkages. Polymers where the units are linked by covalent bonds will be most common but those that include hydrogen bonded units are also embraced by the invention.

The polymer is made up of a plurality of individual units. An “individual unit” as used herein is a building block or monomer which can be linked directly or indirectly to other building blocks or monomers to form a polymer. The polymer preferably is a polymer of at least two different linked units. The at least two different linked units may produce or be labeled to produce different signals.

The methods of the invention can be used to generate information about naturally or non-naturally occurring polymers. This information is based on signals arising from the binding of probes to target polymers. In some instances, the information is unit specific information which refers any structural information about one, some, or all of the units that make up the polymer. If the polymer is a nucleic acid, the units are single nucleotides or combinations of nucleotides, preferably arranged contiguously. The structural information obtained by analyzing a polymer may include the identification of its characteristic properties which (in turn) allows for, for example, determination of its presence or absence in a sample, determination of the relatedness of more than one polymers, determination of the polymer size, determination of the proximity, distance between or order of two or more individual units within a polymer, and/or identification of the general composition of the polymer. Since the structure and function of polymers are generally interdependent, structural information can reveal important information about the function of the polymer.

The sensitivity of methods provided herein allows single polymers such as nucleic acids to be analyzed individually. Analyzing a target polymer generally requires contacting the target polymer with a probe and determining the binding profile or binding pattern of the probe to the target. Binding profiles may simply indicate whether one or more probes are bound to the target. Alternatively, binding profiles may indicate the location of sites within the target which are bound by a probe (thereby providing a map of sites along the target).

The term “nucleic acid” refers to multiple linked nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). “Nucleic acid” and “nucleic acid molecule” are used interchangeably and refer to oligoribonucleotides as well as oligodeoxyribonucleotides. The terms shall also include polynucleosides (i.e., a polynucleotide minus a phosphate) and any other organic base containing nucleic acid. The nucleic acids may be single or double stranded. The nucleic acid being analyzed and/or labeled is referred to as the nucleic acid target.

Nucleic acid targets and nucleic acid probes may be DNA or RNA, although they are not so limited. DNA may be genomic DNA such as nuclear DNA or mitochondrial DNA. RNA may be mRNA, miRNA, siRNA, rRNA and the like. Nucleic acids may be naturally occurring such as those recited above, or may be synthetic such as cDNA. In important embodiments, the nucleic acid is a genomic nucleic acid. In related embodiments, the nucleic acid is a fragment of a genomic nucleic acid. The size of the nucleic acid is not critical to the invention and it is generally only limited by the detection system used.

Harvest and isolation of nucleic acids are routinely performed in the art and suitable methods can be found in standard molecular biology textbooks. (See, for example, Maniatis' Handbook of Molecular Biology.) The nucleic acid may be harvested from a biological sample such as a tissue or a biological fluid. The term “tissue” as used herein refers to both localized and disseminated cell populations including, but not limited, to brain, heart, breast, colon, bladder, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include saliva, sperm, serum, plasma, blood and urine, but are not so limited. Both invasive and non-invasive techniques can be used to obtain such samples and are well documented in the art.

The methods of the invention may be performed in the absence of prior nucleic acid amplification in vitro. In some preferred embodiments, the nucleic acid is directly harvested and isolated from a biological sample (such as a tissue or a cell culture), without amplification. Accordingly, some embodiments of the invention involve analysis of “non in vitro amplified nucleic acids”. As used herein, a “non in vitro amplified nucleic acid” refers to a nucleic acid that has not been amplified in vitro using techniques such as polymerase chain reaction or recombinant DNA methods. A non in vitro amplified nucleic acid may, however, be a nucleic acid that is amplified in vivo (e.g., in the biological sample from which it was harvested) as a natural consequence of the development of the cells in the biological sample. This means that the non in vitro nucleic acid may be one which is amplified in vivo as part of gene amplification, which is commonly observed in some cell types as a result of mutation or cancer development.

In some embodiments, the invention embraces nucleic acid derivatives as targets and/or probes. As used herein, a “nucleic acid derivative” is a non-naturally occurring nucleic acid. Nucleic acid derivatives may contain non-naturally occurring elements such as non-naturally occurring nucleotides and non-naturally occurring backbone linkages. These include substituted purines and pyrimidines such as C—S propyne modified bases, 5-methylcytosine, 2-aminopurine, 2-amino-6-chloropurine, 2,6-diaminopurine, hypoxanthine, 2-thiouracil and pseudoisocytosine. Other such modifications are well known to those of skill in the art.

The nucleic acids may also encompass substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus, modified nucleic acids may include a 2′-O-alkylated ribose group. In addition, modified nucleic acids may include sugars such as arabinose instead of ribose.

The nucleic acids may be heterogeneous in backbone composition thereby containing any possible combination of nucleic acid units linked together such as peptide nucleic acids (which have amino acid linkages with nucleic acid bases, and which are discussed in greater detail herein). In some embodiments, the nucleic acids are homogeneous in backbone composition. Nucleic acids where the units are linked by covalent bonds will be most common but those that include hydrogen bonded units are also embraced by the invention. It is to be understood that all possibilities regarding nucleic acids appear equally to nucleic acid targets and nucleic acid probes.

A nucleic acid target can be bound by one or more sequence specific probes. “Sequence specific” when used in the context of a probe for a nucleic acid target means that the probe recognizes a particular linear arrangement of nucleotides or derivatives thereof. In preferred embodiments, the probe is itself composed of nucleic acid elements such as DNA, RNA, PNA and LNA elements and combinations thereof (as discussed below). In preferred embodiments, the linear arrangement includes contiguous nucleotides or derivatives thereof that each bind to a corresponding complementary nucleotide in the probe. In some embodiments, however, the sequence may not be contiguous as there may be one, two, or more nucleotides that do not have corresponding complementary residues on the probe. The specificity of binding can be manipulated in a number of ways including temperature, salt concentration and the like. Those of ordinary skill in the art will be able to determine optimum conditions for a desired specificity.

It is to be understood that any molecule that is capable of recognizing a target nucleic acid with structural or sequence specificity can be used as a nucleic acid probe. In most instances, such probes will be themselves nucleic acid in nature. Also in most instances, such probes will form at least a Watson-Crick bond with the nucleic acid target. In other instances, the nucleic acid probe can form a Hoogsteen bond with the nucleic acid target, thereby forming a triplex. A nucleic acid probe that binds by Hoogsteen binding enters the major groove of a nucleic acid target and hybridizes with the bases located there. Examples of these latter probes include molecules that recognize and bind to the minor and major grooves of nucleic acids (e.g., some forms of antibiotics). In some embodiments, the nucleic acid probes can form both Watson-Crick and Hoogsteen bonds with the nucleic acid target. BisPNA probes, for instance, are capable of both Watson-Crick and Hoogsteen binding to a nucleic acid.

In some embodiments, the nucleic acid probe is a peptide nucleic acid (PNA), a bisPNA clamp, a pseudocomplementary PNA, a locked nucleic acid (LNA), DNA, RNA, or co-nucleic acids of the above such as DNA-LNA co-nucleic acids. In some instances, the nucleic acid target can also be comprised of any of these elements.

PNAs are DNA analogs having their phosphate backbone replaced with 2-aminoethyl glycine residues linked to nucleotide bases through glycine amino nitrogen and methylenecarbonyl linkers. PNAs can bind to both DNA and RNA targets by Watson-Crick base pairing, and in so doing form stronger hybrids than would be possible with DNA or RNA based probes.

PNAs are synthesized from monomers connected by a peptide bond (Nielsen, P. E. et al. Peptide Nucleic Acids Protocols and Applications, Norfolk: Horizon Scientific Press, p. 1-19 (1999)). They can be built with standard solid phase peptide synthesis technology. PNA chemistry and synthesis allows for inclusion of amino acids and polypeptide sequences in the PNA design. For example, lysine residues can be used to introduce positive charges in the PNA backbone. All chemical approaches available for the modifications of amino acid side chains are directly applicable to PNAs.

Several types of PNA designs exist, and these include single strand PNA (ssPNA), bisPNA and pseudocomplementary PNA (pcPNA).

The structure of PNA/DNA complex depends on the particular PNA and its sequence. Single stranded PNA (ssPNA) binds to single stranded DNA (ssDNA) preferably in antiparallel orientation (i.e., with the N-terminus of the ssPNA aligned with the 3′ terminus of the ssDNA) and with a Watson-Crick pairing. PNA also can bind to DNA with a Hoogsteen base pairing, and thereby forms triplexes with double stranded DNA (dsDNA) (Wittung, P. et al., Biochemistry 36:7973 (1997)).

Single strand PNA is the simplest of the PNA molecules. This PNA form interacts with nucleic acids to form a hybrid duplex via Watson-Crick base pairing. The duplex has different spatial structure and higher stability than dsDNA (Nielsen, P. E. et al. Peptide Nucleic Acids, Protocols and Applications, Norfolk: Horizon Scientific Press, p. 1-19 (1999)).

BisPNA includes two strands connected with a flexible linker. One strand is designed to hybridize with DNA by a classic Watson-Crick pairing, and the second is designed to hybridize with a Hoogsteen pairing. The target sequence can be short (e.g., 8 bp), but the bisPNA/DNA complex is still stable as it forms a hybrid with twice as many (e.g., a 16 bp) base pairings overall.

Pseudocomplementary PNA (pcPNA) (Izvolsky, K. I. et al., Biochemistry 10908-10913 (2000)) involves two single stranded PNAs added to dsDNA. One pcPNA strand is complementary to the target sequence, while the other is complementary to the displaced DNA strand.

Locked nucleic acid (LNA) molecules are known in the art. They form hybrids with DNA, which are at least as stable as PNA/DNA hybrids (Braasch, D. A. et al., Chem & Biol. 8(1):1-7(2001)). Commercial nucleic acid synthesizers and standard phosphoramidite chemistry are used to make LNAs. Naturally, most of biochemical approaches for nucleic acid conjugations are applicable to LNA/DNA constructs.

The probes can also be stabilized in part by the use of other backbone modifications. The invention intends to embrace, in addition to the peptide and locked nucleic acids discussed herein, the use of the other backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as O-linkers, amino acids such as lysine (particularly useful if positive charges are desired in the PNA), and the like. Various PNA modifications are known and probes incorporating such modifications are commercially available from sources such as Boston Probes, Inc.

The nucleic acid probes of the invention can be any length ranging from at least 4 nucleotides long to in excess of 1000 nucleotides long. In preferred embodiments, the probes are 5-100 nucleotides in length, more preferably between 5-25 nucleotides in length, and even more preferably 5-12 nucleotides in length. The length of the probe can be any length of nucleotides between and including the ranges listed herein, as if each and every length was explicitly recited herein. Thus, the length may be at least 5 nucleotides, at least 10 nucleotides, at least 15 nucleotides, at least 20 nucleotides, or at least 25 nucleotides. It should be understood that not all residues of the probe need hybridize to complementary residues in the nucleic acid target. For example, the probe may be 50 residues in length, yet only 25 of those residues hybridize to the nucleic acid target. Preferably, the residues that hybridize are contiguous with each other. Similarly, the probe and any nucleic acids to which it binds including those conjugated to magnetic beads for clean-up purposes need not be of the same size.

The probes are preferably single stranded, but they are not so limited. For example, when the probe is a bisPNA it can adopt a secondary structure with the nucleic acid target resulting in a triple helix conformation, with one region of the bisPNA clamp forming Hoogsteen bonds with the backbone of the target and another region of the bisPNA clamp forming Watson-Crick bonds with the nucleotide bases of the target.

The nucleic acid probe hybridizes to a complementary sequence within the nucleic acid target. The specificity of binding can be manipulated based on the hybridization conditions. For example, salt concentration and temperature can be modulated in order to vary the range of sequences recognized by the nucleic acid probes.

The various reagents, reactive groups, and probes may in some instances include a linker molecule. These linkers can be any variety of molecules, preferably non-active, such as nucleotides or multiple nucleotides, straight or branched saturated or unsaturated carbon chains of carbon, phospholipids, and the like, whether naturally occurring or synthetic. Additional linkers include alkyl and alkenyl carbonates, carbamates, and carbamides.

A wide variety of linkers can be used, many of which are commercially available, for example, from sources such as Boston Probes, Inc. (now Applied Biosystems, Inc.). Linkers are not limited to organic linkers, and rather can be inorganic also (e.g., —O—Si—O—, or O—P—O—). Additionally, they can be heterogeneous in nature (e.g., composed of organic and inorganic elements). Essentially any molecule having the appropriate size restrictions and capable of being linked to the various components such as fluorophore and probe can be used as a linker. As used herein, the terms linker and spacer are used interchangeably.

The probes of the invention are usually labeled with a detectable label. A detectable label is a moiety, the presence of which can be ascertained directly or indirectly. Generally, detection of the label involves the creation of a detectable signal such as for example an emission of energy. The label can be detected directly for example by its ability to emit and/or absorb electromagnetic radiation of a particular wavelength. A label can be detected indirectly for example by its ability to bind, recruit and, in some cases, cleave another moiety which itself may emit or absorb light of a particular wavelength (e.g., an epitope tag such as the FLAG epitope, an enzyme tag such as horseradish peroxidase, etc.). Many naturally occurring units of a polymer are light emitting compounds or quenchers, and thus are intrinsically labeled. Guidelines for selecting the appropriate labels, and methods for adding extrinsic labels to polymers are provided in more detail in U.S. Pat. No. 6,355,420 B1.

Generally the detectable label can be selected from the group consisting of directly detectable labels such as a fluorescent molecule (e.g., fluorescein, rhodamine, tetramethylrhodamine, R-phycoerythrin, Cy-3, Cy-5, Cy-7, Texas Red, Phar-Red, allophycocyanin (APC), fluorescein amine, eosin, dansyl, umbelliferone, 5-carboxyfluorescein (FAM), 2′7′-dimethoxy-4′5′-dichloro-6-carboxyfluorescein (JOE), 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), 5-(2′-aminoethyl) aminonaphthalene-1-sulfonic acid (EDANS), 4-acetamido-4′-isothiocyanatostilbene-2, 2′disulfonic acid, acridine, acridine isothiocyanate, r-amino-N-(3-vinylsulfonyl)phenylnaphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumarin 151), cyanosine, 4′, 6-diaminidino-2-phenylindole (DAPI), 5′, 5″-diaminidino-2-phenylindole (DAPI), 5′, 5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2, 2′-disulfonic acid, 4,4′-diisothiocyanatostilbene-2, 2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl) aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron.RTM.Brilliant Red 3B-A), lissamine rhodamine B sulfonyl chloride, rhodamine B, rhodamine 123, rhodamine X, sulforhodamine B, sulforhodamine 101, sulfonyl chloride derivative of sulforhodamine 101, tetramethyl rhodamine, riboflavin, rosolic acid, and terbium chelate derivatives), a chemiluminescent molecule, a bioluminescent molecule, a chromogenic molecule, a radioisotope (e.g., p³² or H³, ¹⁴C, ¹²⁵I and ¹³¹I), an electron spin resonance molecule (such as for example nitroxyl radicals), an optical or electron density molecule, an electrical charge transducing or transferring molecule, an electromagnetic molecule such as a magnetic or paramagnetic bead or particle, a semiconductor nanocrystal or nanoparticle, a colloidal metal, a colloid gold nanocrystal, a nuclear magnetic resonance molecule, and the like.

The detectable label can also be selected from the group consisting of indirectly detectable labels such as an enzyme (e.g., alkaline phosphatase, horseradish peroxidase, β-galactosidase, glucoamylase, lysozyme, luciferases such as firefly luciferase and bacterial luciferase (U.S. Pat. No. 4,737,456); saccharide oxidases such as glucose oxidase, galactose oxidase, and glucose-6-phosphate dehydrogenase; heterocyclic oxidases such as uricase and xanthine oxidase coupled to an enzyme that uses hydrogen peroxide to oxidize a dye precursor such as HRP, lactoperoxidase, or microperoxidase), an enzyme substrate, an affinity molecule, a ligand, a receptor, a biotin molecule, an avidin molecule, a streptavidin molecule, an antigen (e.g., epitope tags such as the FLAG or HA epitope), a hapten (e.g., biotin, pyridoxal, digoxigenin fluorescein and dinitrophenol), an antibody, an antibody fragment, a microbead, and the like.

Fluorophore pairs are two fluorophores that are capable of undergoing FRET to produce or eliminate a detectable signal when positioned in proximity to one another. Examples of donors include Alexa488, Alexa546, BODIPY493, Oyster556, Fluor (FAM), Cy3 and TMR (Tamra). Examples of acceptors include Cy5, Alexa594, Alexa647 and Oyster656. Cy5 can work as a donor with Cy3, TMR or Alexa546, as an example. FRET should be possible with any fluorophore pair having fluorescence maxima spaced at 50-100 nm from each other.

The label may be of a chemical, lipid, carbohydrate, peptide or nucleic acid nature although it is not so limited. Those of ordinary skill in the art will know of other suitable labels for use in the invention.

In some embodiments, all probes are labeled with the same detectable label. In these embodiments, the pattern of binding rather than the identity of each probe provides enough information. In other embodiments, each probe of a specific sequence is labeled with a unique detectable label. In these embodiments, the detectable labels are used to identify sequences within the target polymer.

In some embodiments, the polymer are labeled with detectable moieties that emit distinguishable signals that can all be detected by one type of detection system. For example, the detectable moieties can all be fluorescent labels or radioactive labels. In other embodiments, the polymers are labeled with moieties that are detected using different detection systems. For example, one polymer or unit may be labeled with a fluorophore while another may be labeled with radioactivity.

Analysis of the polymer involves detecting signals from the labels (potentially through the use of a secondary label, as the case may be), and determining the relative position of those labels relative to one another. In some instances, it may be desirable to further label the polymer with a standard marker that facilitates comparing the information so obtained with that from other polymers analyzed. For example, the standard marker may be a backbone label, or a label that binds to a particular sequence of nucleotides (be it a unique sequence or not), or a label that binds to a particular location in the nucleic acid molecule (e.g., an origin of replication, a transcriptional promoter, a centromere, etc.).

One subset of backbone labels for nucleic acids are nucleic acid stains that bind nucleic acids in a sequence non-specific manner. One major class of such stains is backbone labels which bind nucleic acid backbones. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned are commercially available from suppliers such as Molecular Probes, Inc. Still other examples include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -1, -20, -22, -15, -14, -25 (green), SYTO-81, -80, -82, -83, -84, -85 (orange), SYTO-64, -17, -59, -61, -62, -60, -63 (red).

Polymers can be labeled using antibodies or antibody fragments and their corresponding antigen or hapten binding partners. Detection of such bound antibodies and proteins or peptides is accomplished by techniques well known to those skilled in the art. Antibody/antigen complexes are easily detected by linking a label to the antibodies which recognize the polymer and then observing the site of the label. Alternatively, the antibodies can be visualized using secondary antibodies or fragments thereof that are specific for the primary antibody used. Polyclonal and monoclonal antibodies may be used. Antibody fragments include Fab, F(ab)₂, Fd and antibody fragments which include a CDR3 region.

Conjugation of these labels to for example reactive groups and/or probes can be performed using standard techniques common to those of ordinary skill in the art. For example, U.S. Pat. Nos. 3,940,475 and 3,645,090 demonstrate conjugation of fluorophores and enzymes to antibodies.

As used herein, “conjugated” means two entities stably bound to one another by any physicochemical means. It is important that the nature of the attachment is such that it does not substantially impair the effectiveness of either entity. Keeping these parameters in mind, any covalent or non-covalent linkage known to those of ordinary skill in the art is contemplated unless explicitly stated otherwise herein. Noncovalent conjugation includes hydrophobic interactions, ionic interactions, high affinity interactions such as biotin-avidin and biotin-streptavidin complexation and other affinity interactions. Such means and methods of attachment are known to those of ordinary skill in the art.

The detection system will depend upon the type of detectable labels used. Therefore these roughly correlate with the detectable labels discussed herein. There is a number of detection systems known in the art and these include a fluorescent detection system, a confocal laser microscopy detection system, a near field detection system, a chemiluminescent detection system, a chromogenic detection system, a photographic or autoradiographic film detection system, an electrical detection system, a electromagnetic detection system, a charge coupled device (CCD) detection system, an electron microscopy detection system, an atomic force microscopy (AFM) detection system, a scanning tunneling microscopy (STM) detection system, a scanning electron microscopy detection system, an electron density detection system, a refractive index detection system such as a total internal reflection (TIR) detection system, an electron spin resonance (ESR) detection system, and a nuclear magnetic resonance (NMR) detection system.

Other interactions involved in methods of the invention will produce a nuclear radiation signal. As a radiolabel on a polymer passes through the defined region of detection, nuclear radiation is emitted, some of which will pass through the defined region of radiation detection. A detector of nuclear radiation is placed in proximity of the defined region of radiation detection to capture emitted radiation signals. Many methods of measuring nuclear radiation are known in the art including cloud and bubble chamber devices, constant current ion chambers, pulse counters, gas counters (i.e., Geiger-Müller counters), solid state detectors (surface barrier detectors, lithium-drifted detectors, intrinsic germanium detectors), scintillation counters, Cerenkov detectors, to name a few.

Other types of signals generated are well known in the art and have many detection means which are known to those of skill in the art. Some of these include opposing electrodes, magnetic resonance, and piezoelectric scanning tips. Opposing nanoelectrodes can function by measurement of capacitance changes. Two opposing electrodes create an area of energy storage, located effectively between the two electrodes. It is known that the capacitance of such a device changes when different materials are placed between the electrodes. This dielectric constant is a value associated with the amount of energy a particular material can store (i.e., its capacitance). Changes in the dielectric constant can be measured as a change in the voltage across the two electrodes. In the present example, different nucleotide bases or unit specific markers of a polymer may give rise to different dielectric constants. The capacitance changes with the dielectric constant of the unit specific marker of the polymer per the equation: C=KCₒ, where K is the dielectric constant and Cₒ is the capacitance in the absence of any bases. The voltage deflection of the nanoelectrodes is then outputted to a measuring device, recording changes in the signal with time.

Molecules such as but not limited to polymers may be analyzed using a single molecule analysis system (e.g., a single polymer analysis system). A single molecule detection system is capable of analyzing single molecules separately from other molecules. Such a system may be capable of analyzing single molecules linearly (i.e., starting at a point and then moving progressively in one direction or another) and/or, as may be more appropriate in the present invention, in their totality. In certain embodiments in which detection is based predominately on the presence or absence of a signal, linear analysis may not be required. However, there are other embodiments embraced by the invention which would benefit from the ability to linearly analyze molecules (preferably nucleic acids) in a sample. These include applications in which the sequence of the nucleic acid is desired.

A linear polymer analysis system is a system that analyzes polymers in a linear manner (i.e., starting at one location on the polymer and then proceeding linearly in either direction therefrom). As a polymer is analyzed, the detectable labels attached to it are detected in a sequential manner. The signals may form an image of the polymer, from which distances between labels can be determined. The signals may also be viewed in a histogram (signal intensity vs. time), that can then be translated into a map, with knowledge of the velocity of the polymer. It is to be understood that in some embodiments, the polymer is attached to a solid support, while in others it is free flowing. In either case, the velocity of the polymer as it moves past, for example, an interaction station or a detector, will aid in determining the position of the labels, relative to each other and relative to other detectable markers that may be present on the polymer.

Accordingly, the analysis systems useful in the invention may deduce the total amount of label on a polymer, and in some instances, the location of such labels. The ability to locate and position the labels allows these patterns to be superimposed on other genetic maps, in order to orient and/or identify the regions of the genome being analyzed.

An example of a suitable system is the GeneEngine™ (U.S. Genomics, Inc., Woburn, Mass.). The GeneEngine™ system is described in PCT patent applications WO98/35012 and WO00/09757, published on Aug. 13, 1998, and Feb. 24, 2000, respectively, and in issued U.S. Pat. No. 6,355,420 B1, issued Mar. 12, 2002. The contents of these applications and patent, as well as those of other applications and patents, and references cited herein are incorporated by reference in their entirety. This system is both a single molecule analysis system and a linear polymer analysis system. It allows, for example, single nucleic acids to be passed through an interaction station in a linear manner, whereby the nucleotides in the nucleic acid are interrogated individually in order to determine whether there is a detectable label conjugated to the nucleic acid. Another suitable system is the Trilogy™ (U.S. Genomics, Inc., Woburn, Mass.) which is a single molecule analysis system. Interrogation in either system involves exposing the nucleic acid to an energy source such as optical radiation of a set wavelength. The mechanism for signal emission and detection will depend on the type of label sought to be detected, as described herein.

Other single molecule nucleic acid analytical methods which involve elongation of DNA molecules can also be used in the methods of the invention. These include fiber-fluorescence in situ hybridization (fiber-FISH) (Bensimon, A. et al., Science 265(5181):2096-2098 (1997)). In fiber-FISH, nucleic acid molecules are elongated and fixed on a surface by molecular combing. Hybridization with fluorescently labeled probe sequences allows determination of sequence landmarks on the nucleic acid molecules. The method requires fixation of elongated molecules so that molecular lengths and/or distances between markers can be measured. Pulse field gel electrophoresis can also be used to analyze the labeled nucleic acid molecules. Pulse field gel electrophoresis is described by Schwartz, D. C. et al., Cell 37(1):67-75 (1984). Other nucleic acid analysis systems are described by Otobe, K. et al., Nucleic Acids Res. 29(22):E109 (2001), Bensimon, A. et al. in U.S. Pat. No. 6,248,537, issued Jun. 19, 2001, Herrick, J. et al., Chromosome Res. 7(6):409:423 (1999), Schwartz in U.S. Pat. No. 6,150,089 issued Nov. 21, 2000 and U.S. Pat. No. 6,294,136, issued Sep. 25, 2001. Other linear polymer analysis systems can also be used, and the invention is not intended to be limited to solely those listed herein.

Optical detectable signals are generated, detected and stored in a database. The signals can be analyzed to determine structural information about the nucleic acid. The signals can be analyzed by assessing the intensity of the signal to determine structural information about the nucleic acid. The computer may be the same computer used to collect data about the nucleic acids, or may be a separate computer dedicated to data analysis. A suitable computer system to implement embodiments of the present invention typically includes an output device which displays information to a user, a main unit connected to the output device and an input device which receives input from a user. The main unit generally includes a processor connected to a memory system via an interconnection mechanism. The input device and output device also are connected to the processor and memory system via the interconnection mechanism. Computer programs for data analysis of the detected signals are readily available from CCD (charge coupled device) manufacturers.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. All references, patents and patent applications that are recited in this application are incorporated by reference herein in their entirety.

What is claimed is:

1. A method for analyzing a polymer in an unstretched conformation comprising
identifying a single hairpin polymer in a polymer population based on a measured length that is shorter than a contour length, and
comparing a probe binding profile of the single hairpin polymer to a hairpin dataset,
wherein the polymer is labeled with a backbone label.

2. The method of claim 1, wherein the measured length is determined based on transit time of the polymer between two positions and wherein the polymer is traveling at a known velocity.

3. The method of claim 1, wherein the contour length is determined by measuring total backbone label signal intensity for the polymer.

4. The method of claim 1, wherein total backbone label signal intensity is total integrated backbone label signal intensity.

5. A method for analyzing a polymer in an unstretched conformation comprising
determining total integrated backbone label signal intensity of a polymer as an indicator of contour length wherein the polymer is labeled with a backbone label,
determining measured length based on transit time of the polymer between two positions wherein the polymer is traveling at a known velocity,
comparing contour length and measured length wherein a measured length that is shorter than a contour length is indicative of an unstretched polymer,
determining a probe binding profile of the unstretched polymer, and
processing the probe binding profile.

6. The method of claim 5, wherein processing the probe binding profile comprises derivation of de novo sequence information from the profile.

7. The method of claim 5, wherein processing the probe binding profile comprises comparing the probe binding profile to a hairpin dataset.

8. The method of claim 1, wherein the hairpin dataset contains forward and reverse orientation probe binding profiles for a hairpin polymer.

9. The method of claim 1, further comprising re-orienting probe binding profiles according to leading edge or trailing edge high signal intensity backbone regions.

10. The method of claim 1, wherein the polymer is a nucleic acid.

11. The method of claim 10, wherein the nucleic acid is a DNA or RNA.

12. The method of claim 1, wherein the probe binding profile is a sequence specific probe binding profile.

13. The method of claim 12, wherein sequence specific probe binding profile is generated using sequence specific probes that are identical in sequence and label.

14. The method of claim 12, wherein sequence specific probe binding profile is generated using sequence specific probes that differ in sequence and label.

15. The method of claim 12, wherein sequence specific probe binding profile is generated using at least one sequence specific probe that is unique to an organism.

16. The method of claim 12, wherein sequence specific probe binding profile is generated using sequence specific probes that are chosen to yield a unique probe binding profile indicative of an organism.

17. The method of claim 1, wherein the hairpin dataset is a dataset of signals from hairpin configurations of pathogen derived polymers.

18. The method of claim 17, wherein the hairpin dataset comprises signals from hairpin configurations of polymers from a single pathogen.

19. The method of claim 17, wherein the pathogen derived polymers are derived from biohazardous pathogens.

20. The method of claim 1, wherein the hairpin dataset comprises signals from hairpin configurations of human derived polymers.

21. The method of claim 20, wherein the human derived polymers are genomic DNA.
